(19) United States
(12) Patent Application Publication
(10) Pub. No.: US 2012/0116256 A1
(43) Pub. Date: May 10, 2012

(54) MEASUREMENT OF BIO-SIGNALS

(75) Inventors: Oyvind Stavdahl, Heimdal (NO); Anders Lyngvi Fougner, Trondheim (NO); Peter Joseph Kyberd, Fredericton (CA)

(73) Assignee: NTNU TECHNOLOGY TRANSFER AS, Trondheim (NO)

(21) Appl. No.: 13/057,904

(22) PCT Filed: Aug. 7, 2009
(86) PCT No.: PCT/GB09/01956

§ 371 Date: May 2, 2011

Related U.S. Application Data
(60) Provisional application No. 61/087,480, filed on Aug. 8, 2008.

(30) Foreign Application Priority Data
Aug. 8, 2008 (GB) 0814533.6

(51) Int. Cl. A61B 5/11

(52) U.S. Cl. 600/595

(57) ABSTRACT

Apparatus for measuring a bio-signal associated with contraction of a muscle in a human or animal body. The apparatus has a sensor for placement in contact with the body for measuring said muscle contraction signal. It also includes means for measuring a contact force and/or relative movement between the sensor and the body.

This invention relates to the measurement of signals generated by a biological entity for controlling an artificial device: particularly, although not exclusively, to controlling a prosthesis, orthosis or another device being controlled through a myoelectric, myoacoustic or similar control interface.

People who suffer the loss of a limb are frequently offered a “replacement limb”, or prosthesis. The prosthesis may be completely passive and serve mainly cosmetic purposes, but it may also incorporate mechanical joints actuated by the user through a harness arrangement. Perhaps even more frequently, the prosthesis comprises mechanical joints actuated by electrical motors. These motors are controlled by an electronic control system on the basis of various control signals issued by the user. The state-of-the-art in upper-limb prosthesis control is using myoelectric signals, generated by activated muscles, recorded by electrodes resting on the skin surface, for controlling the prosthesis. The amplitude of the myoelectric signal is crudely proportional to the level of contraction of the underlying muscle. By detecting the amplitude of such a signal or set of signals, and thus the level of contraction of the related muscle(s), the control system can predict the motor intent of the user and control the movements of the prosthesis accordingly.

Numerous examples exist of different variations of such a system, one being disclosed by Gammer et al. (CA 2148577). This document describes a system in which the user can control joint velocity and gripping force of a hand prosthesis proportionally by varying the amplitude of a myoelectric signal being measured on the user's skin surface.

Surface myoelectric signals are detected by electrodes that are in physical contact with the skin in the vicinity of the muscle in question, and are usually pre-amplified before being conveyed to a control system for further processing. As with most other bio-electrical signals, the myoelectric signal is notoriously susceptible to electromagnetic disturbances, or unwanted artifacts, in the measured signal. The effect of these artifacts is that they cause changes in the myoelectric signal that are erroneously interpreted by the control system as a change in muscle contraction level or pattern, and thus causing the prosthesis to behave different from what the user solicits. The significance of this problem increases as modern prostheses exhibit an increased number of controllable joints and consequently require more complex and still more precise control systems.

It has previously been proposed by Smits (U.S. Pat. No. 5,711,307) to estimate continuously the amount of noise in the signals detected, and turn the signal “off” whenever the detected noise level exceeds a preset threshold. However this is not ideal in a real-time control system, particularly in the context of a prosthesis which a user would then be unable to control.

The Applicant has recognised that surface myoelectric signal also is highly susceptible to artifacts caused by varying levels of moisture (i.e. sweat build up around the electrodes) as well as relative movements and variations in the contact force (including normal force, shear and tear force, torques and more) between the electrodes and underlying tissue. A previous proposal has been made to estimate the effect of varying moisture in order to cancel the accompanying signal artifact (G. C. Ray and S. K. Guha, “Equivalent electrical representation of the sweat layer and gain compensation of the EMG amplifier.” IEEE Trans Biomed Eng 1983 February; 30(2): 130-2). However this does not fully address the problem.

When viewed from a first aspect the invention provides an apparatus for measuring a bio-signal associated with contraction of a muscle in a human or animal body comprising a sensor for placement in contact with said body for measuring said muscle contraction signal and further comprising means for measuring in use a contact force and/or relative movement between said sensor and said body.

When viewed from a second aspect the invention provides a method of measuring a bio-signal associated with contraction of a muscle in a human or animal body comprising using a sensor to carry out a measurement of said bio-signal and measuring a contact force and/or relative movement between said sensor and said body.

Thus in accordance with the aspects of the invention set out above, a signal associated with muscle contraction can be measured more accurately by taking account of the contact force and/or relative movement between the sensor and the body which in turn allows the artifacts introduced into the measured signal to be compensated for. This has been found to give more accurate and reliable control over an active prosthesis.

In preferred embodiments the means for measuring the contact force and/or relative movement comprises at least one additional sensor. In some preferred embodiments a plurality of such sensors is provided. In one non-limiting example force sensing resistors are used. Preferably at least one of the additional sensors is located adjacent the bio-signal sensor. Preferably the means for measuring the contact force and/or relative movement is provided on a common support with the bio-signal sensor. Preferably the means for measuring the contact force and/or relative movement is mounted in a fixed relationship with the bio-signal sensor. When viewed from a further aspect the invention provides an interface apparatus for providing an interface between a human or animal body and an artificial device, said interface apparatus comprising a first, bio-signal sensor and a second sensor for measuring the contact force and/or relative movement between the first sensor and the body, wherein said first and second sensors are provided on a common support.

The bio-signal specified in accordance with the invention could be any signal associated with a function of a biological entity. The term signal is not here limited to any particular type of signal, but encompasses any externally measurable quantity within or emanating from a biological entity. The bio-signal may be naturally-occurring, or may be artificially generated; for example, by an implant arranged to emit a signal (e.g. a radio-frequency signal) in response to a stimulus (e.g. a myoelectric signal). In one set of preferred embodiments the bio-signal is a myoelectric signal (an electrical signal produced by a muscle as it contracts).

In another set of embodiments myoacoustic signals could be used. This concept exploits the acoustic sound produced by a muscle during contraction. Myoacoustic signals are quite similar to myoelectric signals, except that they are picked up by a microphone instead of a set of electrodes, and are susceptible to acoustic noise rather than electromagnetic noise. The problems associated with motion and contact force are quite similar to those encountered in conjunction with myoelectric control. Given the fact that acoustic signals are mechanical pressure waves travelling through a medium, a particular set of embodiments could use a single sensor element for recording both the relatively high-frequency myoacoustic signal and the more low-frequency contact force signal.

In a further set of embodiments the bio-signal could be a neuroelectric signal (the nerve impulses used to control muscles). The Applicant has appreciated that these exhibit most of the properties of myoelectric signals, and consequently lend themselves to use in control interfaces of the kind described herein.

The invention is not limited to using just one signal—any number or combination could be used.

The principles of the invention could be applied more widely than just measuring muscle contraction and thus any other type of bio-signals generated from a biological entity could be used. Accordingly when viewed from a further aspect the invention provides a method of measuring a bio-signal generated from a biological entity comprising using a sensor to carry out a measurement of said bio-signal and measuring a contact force and/or relative movement between said sensor and said body. The invention extends to corresponding apparatus adapted and suitable for carrying out this method.

The contact force and/or relative movement could, for example, include stretching of the skin, removal of the sensor relative to the skin, shear force or rotation. Relative movement between the sensor and the body should be understood as meaning relative movement between the sensor and a part of the body where sensing is taking place; e.g. between the sensor and skin with which the sensor is in contact, or between the sensor and a muscle or bone closest to the sensor. It is not intended to cover relative movement between a sensor attached to one part of a human body (e.g. an arm) and a different part of the same body (e.g. a foot).

Equally there may be other factors which give rise to unwanted artifacts in the measured bio-signal. For example body temperature (which influences sweat and body heat) or moisture, In more general terms it may be seen that when measuring muscle contraction there are a variety of bio-signals from which information can be derived about muscle contraction or artifacts which affect measurements thereof, such as: myoelectric, myoacoustic or neuroelectric signals; sensor contact, shear or rotational force or acceleration; temperature, moisture. By measuring combinations of these, a more accurate estimate of muscle contraction can be achieved than by measuring one alone, Thus when viewed from another aspect the invention provides a method of estimating muscle contraction comprising measuring two different bio-signals and calculating an estimate from muscle contraction from a combination of both signals.

The invention also extends to a method of measuring a bio-signal generated from a biological entity comprising using a first sensing means to carry out a measurement of said signal and using a second sensing means to measure a parameter affecting said measurement other than the bio-signal.

In preferred embodiments the bio-signal is associated with a muscle contraction.

The invention also extends to a method of controlling an artificial device comprising using a bio-signal measured in accordance with any of the foregoing methods or with any of the foregoing apparatus. Indeed, when viewed from a further aspect the invention provides a prosthesis having control means comprising a sensor adapted to measure a bio-signal associated with contraction of a muscle and means for measuring a parameter affecting said measurement other than the bio-signal.

The nature of the device controlled in accordance with the invention is not limited however. Thus while the discussion above has focussed on controlling a prosthesis, the muscle contractions or other bio-signals could be used to control any machine—whether or not the machine has movements that can be correlated with the muscle movements—e.g. it could be used to control the physical movement of a wheelchair, or the movement of a cursor on a screen, or a selector for a text editor or speech synthesiser.

An experimental implementation of the invention will now be described, by way of example only, with reference to the accompanying Figures in which:

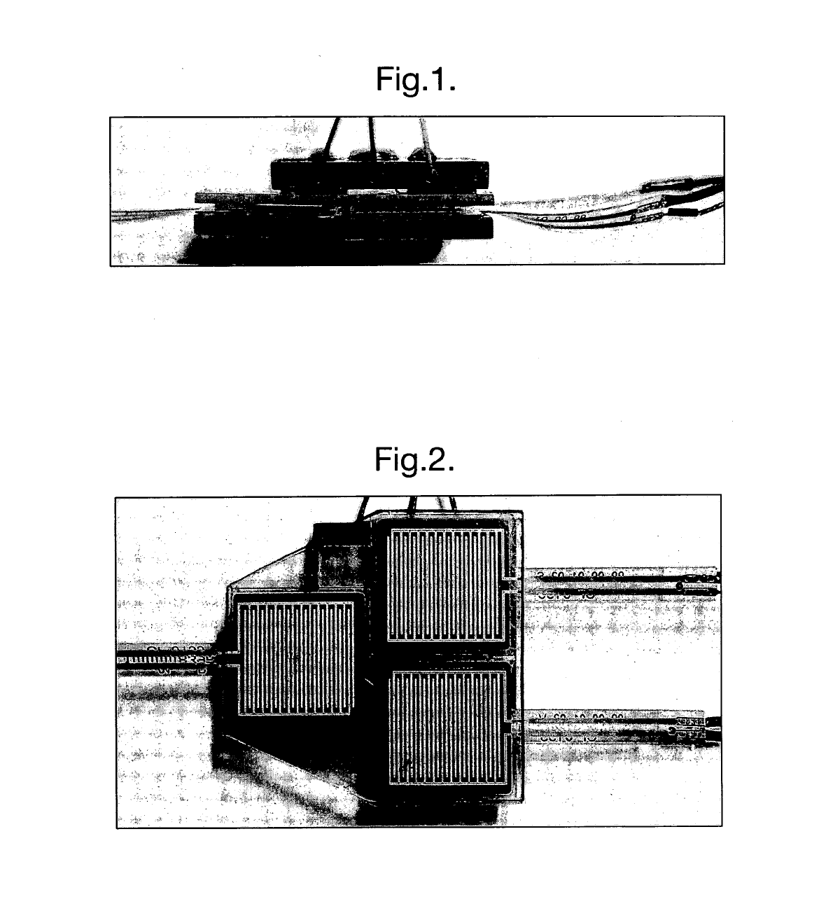

FIG. 1 is a photograph showing a first view of a sensor structure for an experimental set-up;

FIG. 2 is a photograph showing a second view of the sensor structure;

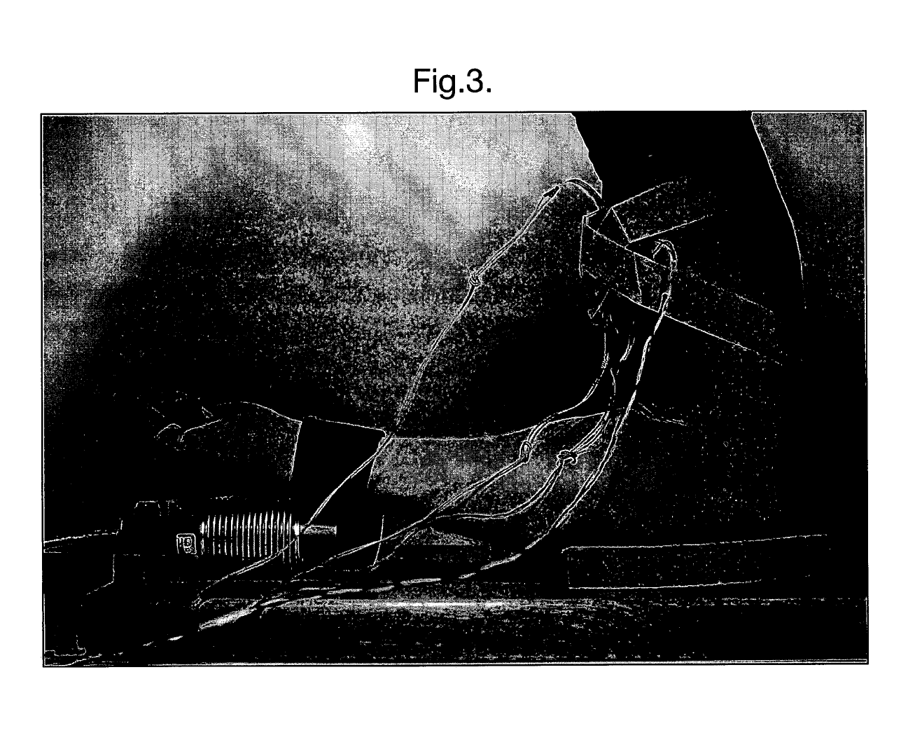

FIG. 3 is a photograph showing the experimental set-up;

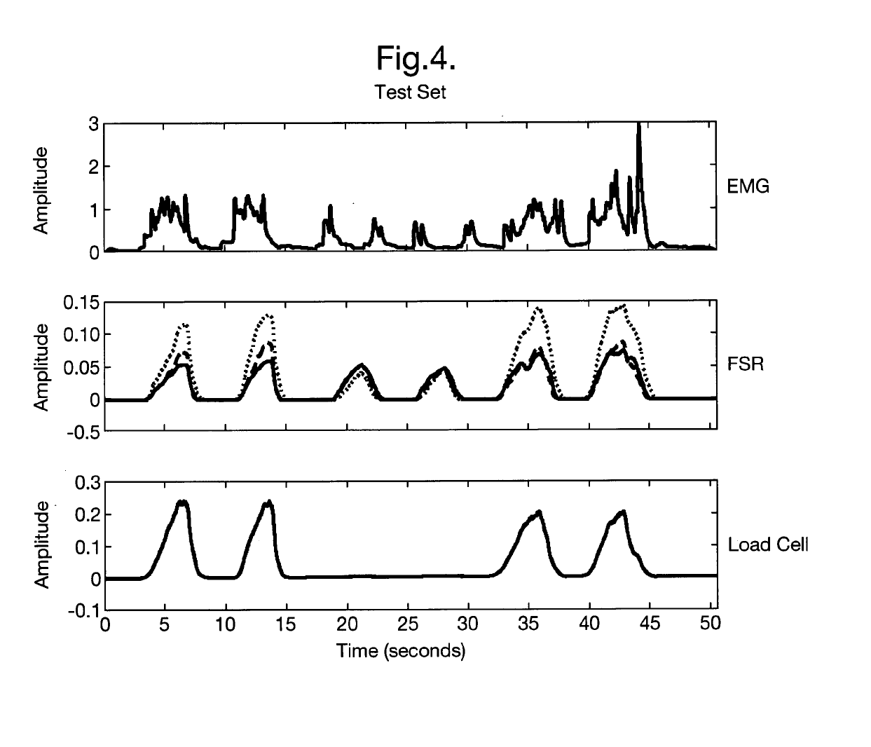

FIG. 4 is a series of graphs showing the signals measured by different sensors during the experiments;

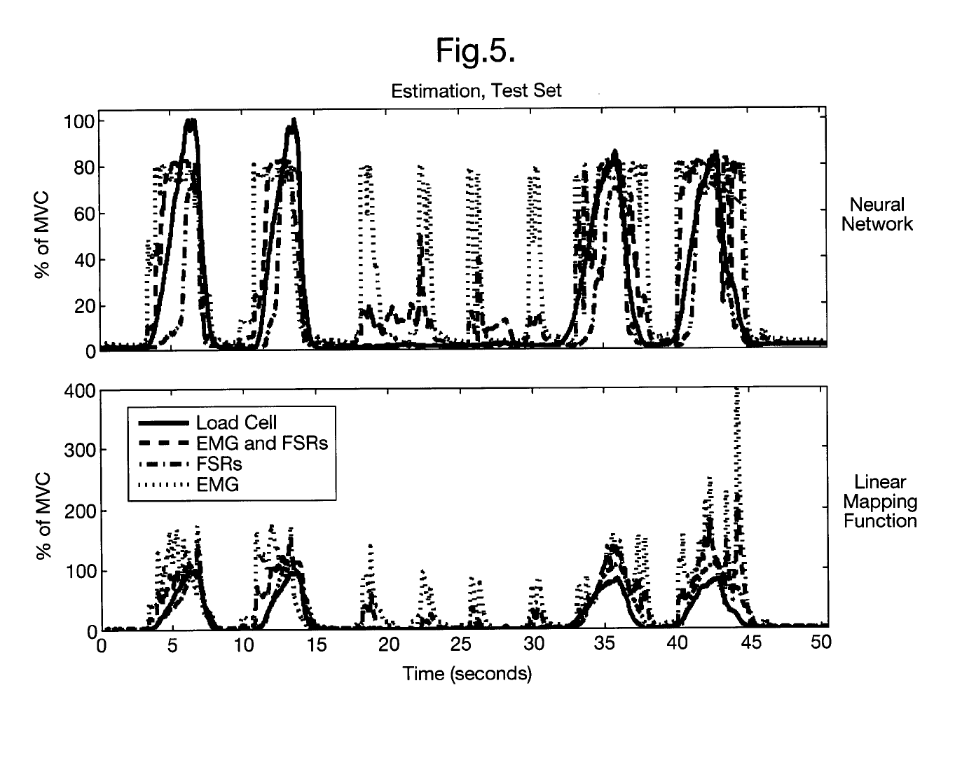

FIG. 5 is a graph of estimation results for three different test set inputs;

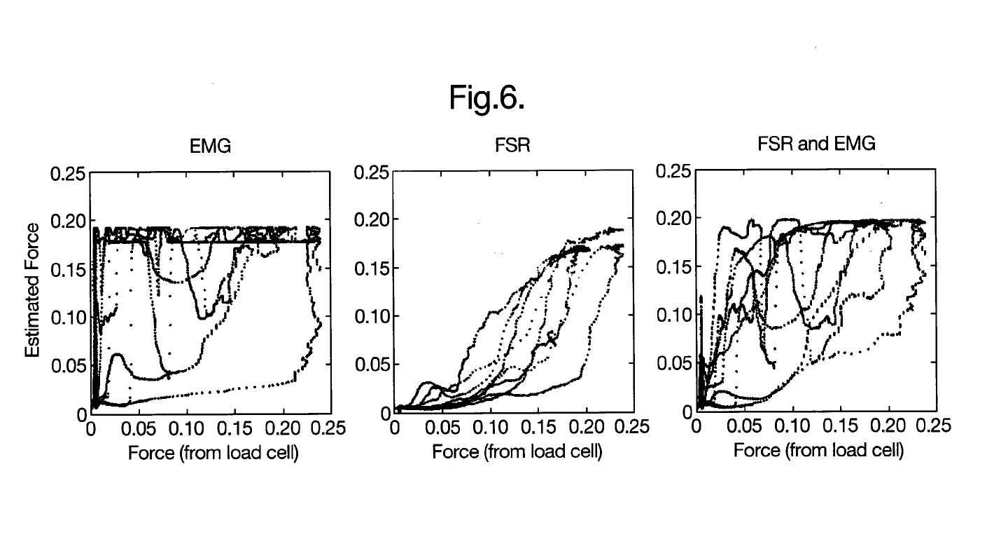

FIG. 6 is a series of plots of measured vs estimated contraction force; and

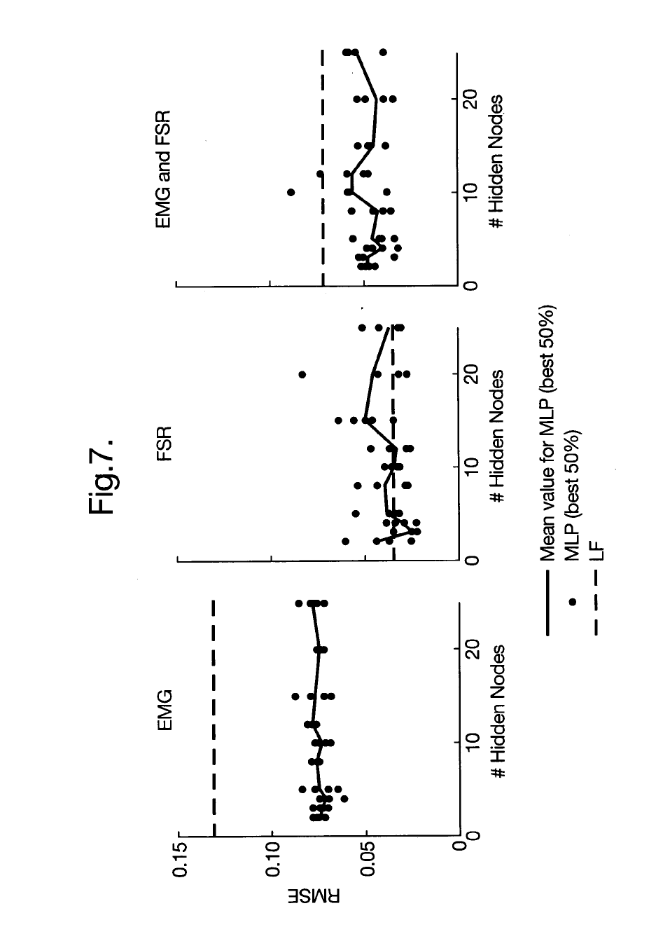

FIG. 7 is a plot of RMS error rates for the data sets of FIGS. 3 and 4.

A laboratory test set-up was constructed comprising a surface myoelectrogram (sEMG) sensor unit built from the metal electrodes of an Otto Bock 13E125 device, mounted with the original spacing and wired to an external preamplifier. Three force sensing resistors (FSRs) were used as the force sensors. FSRs were used for their flatness and simplicity of use. Three individual FSRs allow both magnitude and position/direction of an external force to be estimated, factors both of which could be relevant for the identifying unwanted artifacts in the primary signal. The FSR sensors depicted in the experimental set up of FIGS. 1-3 are off-the-shelf components but smaller sensors could be constructed so that the entire device will fit into a prosthesis socket.

The sensors were sandwiched between two layers of acrylic glass using soft double sided tape (FIGS. 1 and 2). The electrodes were attached to this structure with the reference electrode at the centre of the FSR array.

The device was taped to the m. biceps brachii of a healthy subject and tested by simultaneously measuring sEMG and FSR outputs while muscle contraction force was measured using a load cell (FIG. 3). The sEMG signal was pre-processed with a non-linear myoprocessor as described in Fougner, A., “Proportional Myoelectric Control of a Multifunction Upper-limb Prosthesis”, Master's Thesis, Norwegian University of Science and Technology, Norway, 2007. External forces in random directions were applied to the sensor during the measurements in order to induce artifacts. Data was collected at 218 Hz for approx. 50 s. Three data sets were acquired; a training set and a validation set collected immediately after each other, and a test set acquired after having removed and then reapplied the device to the subject's arm.

Multilayer perceptron (MLP) networks with different numbers of hidden nodes (2-25 nodes, 10 MLP networks of each size) were employed to estimate the muscle force based on sEMG and FSR signals. Following MLP training and validation, the best 50% of the MLP networks of each size were chosen for final assessment using the test set. A linear and a quadratic mapping function were also fitted to the training set for comparison.

### Results Obtained

FIG. 4 presents an example data set with all recorded data. Note the two central peaks in the FSR signals, which are not accompanied by peaks in the load cell signal; these represent artifacts. The result of the force estimation, using the test set and an MLP network and a linear mapping function, respectively, is presented in FIG. 5.

FIG. 6 shows the estimated against measured force for the test set after training and validating the MLP network. Note the presence of hysteresis in the FSR based estimate; this is caused by intrinsic properties of the FSR sensors, and can be eliminated by using another force sensor technology. Also note the presence of force artifacts in both sEMG based graphs, evident as significant force estimate values at approximate zero load cell force.

The root mean square error (RMSE) rates for the different combinations of sEMG and FSR as inputs are presented in FIG. 7. No reduction in RMSE was detected when increasing the number of hidden MLP nodes beyond n=4.

It is noted that in FIG. 5 and FIG. 6, the FSR based estimates exhibit little or no artifact from external forces, which is at first a little surprising. In FIG. 4, however, it can be seen that the pure disturbance (i.e. the middle two “peaks”) cause an equal response in all three FSRs, while when the muscle actually contracts, the FSRs yield different signal levels. Consequently, the estimator is able to distinguish these two signal sources.

In the upper graph of FIG. 4, the processed sEMG exhibits a transient response to the disturbance. This suggests that an optimal contraction force estimator should have a dynamic aspect rather than a purely static mapping property like the ones investigated in this study.

It will be appreciated by those skilled in the art that the experimental set up described above is purely exemplary and that many aspects thereof would be different in a practical implementation of the invention in a prosthesis or the like. The nature of the sensors employed in practice will influence the precise nature of the artifacts generated and the parameters used to compensate for them. However all such possibilities maintain the central principles of the invention and are encompassed within it.

1. An apparatus for measuring a bio-signal associated with contraction of a muscle in a human or animal body comprising:
a first sensor for placement in contact with said body for measuring said muscle contraction signal; and
a sensor arrangement for measuring in use a contact force and/or relative movement between said first sensor and said body.

2. The apparatus of claim 1, wherein said sensor arrangement for measuring in use a contact force and/or relative movement between said first sensor and said body comprises at least one additional sensor.

3. The apparatus of claim 1, wherein said sensor arrangement for measuring in use a contact force and/or relative movement between said first sensor and said body comprises a force sensing resistor.

4. The apparatus of claim 1, wherein said sensor arrangement for measuring in use a contact force and/or relative movement between said first sensor and said body is provided on a common support with the first sensor.

5. The apparatus of claim 1, wherein said sensor arrangement for measuring in use a contact force and/or relative movement between said first sensor and said body is mounted in a fixed relationship with said first sensor.

6. An interface apparatus for providing an interface between a human or animal body and an artificial device, said interface apparatus comprising:
a first bio-signal sensor; and
a second sensor for measuring the contact force and/or relative movement between the first sensor and the body, wherein said first and second sensors are provided on a common support.

7. The interface apparatus of claim 6, wherein the bio-signal sensor is adapted to sense a bio-signal associated with a muscle contraction.

8. The interface apparatus of claim 6, wherein said second sensor is mounted in a fixed relationship with the bio-signal sensor.

9. The interface apparatus of claim 6, wherein the bio-signal sensor is adapted to sense a myoelectric signal.

10. The interface apparatus of claim 6, wherein the bio-signal sensor is adapted to sense a myoacoustic signal.

11. The interface apparatus of claim 10, further comprising a single sensor element for recording both the myoacoustic signal and the contact force and/or relative movement signal.

12. The interface apparatus of claim 6, wherein the bio-signal sensor is adapted to sense a neuroelectric signal.

13. An apparatus for measuring a signal associated with contraction of a muscle in a human or animal body comprising:
A first sensor for placement in contact with said body for measuring said muscle contraction signal; and
a sensor arrangement for measuring in use a contact force and/or relative movement between said first sensor and said body.

14. A method of measuring a bio-signal associated with contraction of a muscle in a human or animal body comprising:
using a sensor to carry out a measurement of said bio-signal; and
measuring a contact force and/or relative movement between said sensor and said body.

15. The method of claim 14, wherein the bio-signal is associated with a muscle contraction.

16. The method of claim 14, wherein the bio-signal is a myoelectric signal.

17. The method of claim 14, wherein the bio-signal is a myoacoustic signal.

18. The method of claim 17, further comprising using a single sensor element for recording both the myoacoustic signal and the contact force signal.

19. The method of claim 14, wherein the bio-signal is a neuroelectric signal.

20. A method of measuring a bio-signal generated from a biological entity comprising:
using a sensor to carry out a measurement of said bio-signal; and
measuring a contact force and/or relative movement between said sensor and said body.

21. The method of claim 20, wherein the step of measuring said contact force and/or relative movement comprises measuring a force or movement selected from the group consisting of stretching of the skin, removal of the sensor relative to the skin, shear force, and rotation.

22. A method of estimating muscle contraction comprising:
measuring two different bio-signals; and
calculating an estimate of muscle contraction from a combination of both signals.

23. A method of measuring a bio-signal generated from a biological entity comprising:
using a first sensor to carry out a measurement of said signal, and
using a second sensor to measure a parameter affecting said measurement other than the bio-signal.

24. An apparatus adapted to carry out a method of measuring a bio-signal associated with contraction of a muscle in a human or animal body comprising:
using a sensor to carry out a measurement of said bio-signal; and
measuring a contact force and/or relative movement between said sensor and said body.

25. A method of controlling an artificial device comprising:
using an interface apparatus for providing an interface between a human or animal body and the artificial device, said interface apparatus comprising a first bio-signal sensor and a second sensor for measuring the contact force and/or relative movement between the first sensor and the body, wherein said first and second sensors are provided on a common support.

26. A method of controlling an artificial device comprising:
using a bio-signal associated with contraction of a muscle in a human or animal body;
using a sensor to carry out a measurement of said bio-signal; and
measuring a contact force and/or relative movement between said sensor and said body.

27. A prosthesis having control means comprising:
a sensor adapted to measure a bio-signal associated with contraction of a muscle; and
one or more sensors for measuring a parameter affecting said measurement other than the bio-signal.
